(19) United States
(12) Patent Application Publication
(10) Pub. No.: US 2006/0025730 A1
(43) Pub. Date: Feb. 2, 2006

(54) METHOD AND APPARATUS FOR CAPTURING DEBRIS GENERATED FROM A PROCEDURE ON A HUMAN'S EXTREMETIES

(76) Inventor: William J. Hartigan JR., Arlington, MA (US)

Correspondence Address: MESMER & DELEAULT, PLLC, 1 NEW HAMPSHIRE AVE., SUITE 125, PORTSMOUTH, NH, 03801, US

(21) Appl. No.: 11/163,373

(22) Filed: Oct. 17, 2005

Related U.S. Application Data
(63) Continuation-in-part of application No. 10/707,326, filed on Dec. 5, 2003.
(60) Provisional application No. 60/319,758, filed on Dec. 5, 2002.

(51) Int. Cl. A61M 1/00

(52) U.S. Cl. 604/322

(57) ABSTRACT

A method and apparatus for capturing debris generated from a procedure on a human's extremities having a support bar that can be removably secured to an item of furniture, a flexible or rigid frame secured to the support bar adapted to receive a flexible receptacle, and at least one flexible receptacle placed upon or removably secured to the frame. Procedures include, but are not limited to, podiatry, manicure, and pedicure.

### BACKGROUND

1. Field of the Invention

The invention is related to surgical, body protecting, or restraining devices for patients; and folded or stacked surgical drapes; and nail care procedures.

2. Description of the Related Art

Two of the most important tools used by a foot doctor and nail care practitioner are the chair and collection receptacle for foot-related debris. They are often used in close proximity to each other to enable the practitioner to work efficiently and hygienically. In use, the patient or client sits in the chair and extends his leg over a receptacle for catching tissue, dirt, nails, wipes, gauze, and other disposable items used in podiatry or the field of nail care. It is important to capture this material, not only for cleanliness, but to prevent the spread of disease.

Several chair and receptacle arrangements have been used in the past. One such arrangement is disclosed in U.S. Pat. No. 2,519,771, which issued to Lacore in 1947 (the '771 patent). The '771 patent is not admitted to being prior art by its mention in this Background section. The '771 patent discloses a chiropodist's chair with a rigid, removable waste receptacle. The receptacle is removable by sliding it out of its holder, and then it must be emptied and cleaned. This device still requires the podiatrist or nail care practitioner to expose himself to foot waste and associated debris, and it does not cover the practitioner's lap and legs.

Similarly, a podiatry chair having a slideably removable receptacle is disclosed in U.S. Pat. No. 4,852,941, which issued to Jones in 1989 (the '941 patent). The '941 patent is not admitted to being prior art by its mention in this Background section. Like the '771 patent disclosure, this apparatus also has a rigid receptacle that must be emptied and cleaned, exposing the podiatrist or pedicure practitioner to foot waste and associated debris.

Some have tried to avoid these shortcomings by placing a towel, such as a paper towel, in the rigid receptacle and on the floor to catch the debris. This method also has some drawbacks. The towels are not secured in any effective way, and can be accidentally moved from their desired position. The area covered by the towels is often inadequate, resulting in debris landing in areas not covered by the towels, usually on the floor, or the leg or the lap of the practitioner.

Another solution is described in U.S. Pat. No. 4,955,666, which issued to Baker in 1999 (the '666 patent). The '666 patent is not admitted to being prior art by its mention in this Background section. The '666 patent describes a chair and debris-catching receptacle primarily for use in gynecological examinations. Here, the receptacle is disposable, but is secured very closely to the chair by a hoop, which is therefore better suited for gynecological rather than podiatric examination or nail care. It would not be practical to adapt this invention to a podiatric examination method and apparatus, which would require a hoop secured to the chair seat extended beyond the length of the patient's legs and feet. In addition, the above apparatuses are permanent parts of the chairs, which make them expensive and not very portable. What is needed, therefore, is a method and apparatus for capturing debris generated from a procedure on a human's extremities that employs a disposable receptacle and can be removed from the chair when not in use.

### SUMMARY

A method and apparatus for capturing debris generated from a procedure on a human's extremities comprises a support bar that can be removably secured to a chair, a frame adjustably secured to the support bar adapted to receive a disposable receptacle, and at least one receptacle removably secured to the frame. These and other features, aspects, and advantages of the present invention will become better understood with regard to the following description, claims, and accompanying drawings.

### DRAWINGS

FIG. 1 is a plan view of an apparatus of the present invention without a receptacle installed.

FIG. 2 is a side elevation of the apparatus of FIG. 1.

FIG. 3 is a side elevation of the apparatus of FIG. 1 with a receptacle installed.

FIG. 4 is a side elevation of the apparatus of FIG. 2 used with a stool.

FIG. 5 is a side elevation of the apparatus of FIG. 2 secured to height-adjustable support legs.

FIG. 6 is a side elevation of the apparatus of FIG. 2 removably secured to a podiatric chair.

FIG. 7 is a plan view of another embodiment having a swivel frame.

FIG. 8 is a side elevation of the apparatus of FIG. 7.

FIG. 9 is a side elevation of the apparatus of FIG. 7 with a receptacle installed.

FIG. 10 is a side elevation of the apparatus of FIG. 8 removably secured to a podiatric chair.

FIG. 11 is a side elevation of the apparatus directly secured to a chair without a support bar.

### DESCRIPTION

In use, the method and apparatus involves using a flexible, disposable receptacle in place of using a rigid receptacle and optionally two or three towels as had been done previously. The receptacle of the present invention covers the area under the patient's or client's legs as well as below his feet and over the legs and lap of the practitioner. Typically, the receptacle would cover an area of about two to three feet in diameter. The receptacle would be supported by a frame preferably made of metal wire or plastic, and be generally hoop-shaped. The frame can be flexible and is designed to hold its shape when flexed. In this way, the operator can adapt the frame to the procedure being undertaken. The frame 20 can also be substantially rigid. Any number of receptacles could be placed on the frame, much like one bag inside another, and another, and so on. The frame with receptacles, in one embodiment, would be removably secured to a support bar already removably secured to a chair. The support bar stabilizes the receptacle in relation to a surgical procedure or nail care chair. The support bar could be attached in many different ways, including strapping it onto the chair.

The method of the present invention would generally have the following steps. A frame would be secured to the support bar. At least one receptacle would be removably secured to the frame. The patient or client would sit in the chair, resting his legs on top of the receptacle and frame underneath. The procedure would take place, and the patient or client would then get up from the chair. The receptacle would be removed much like a trash bag from a trash can, and disposed of, and, if more than one receptacle were installed, especially in a cartridge-style embodiment, another fresh receptacle would be left for the next procedure. If a plurality of receptacles were installed, the process is repeated until all the receptacles attached to the frame have been used. If a cartridge-style embodiment was used, the frame would be removed from the support bar and a new frame with receptacles already loaded would be attached. This method could easily be modified to work with an independent support structure, such as a tripod or pedestal, instead of a support bar attaching to an item of furniture.

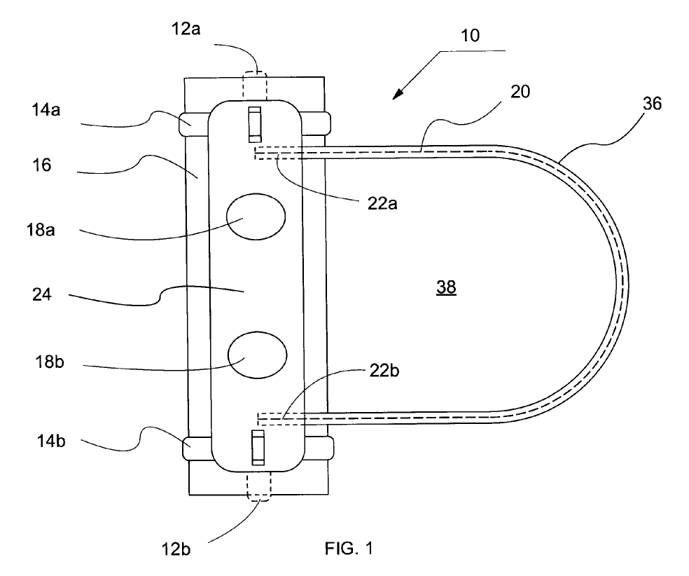

Turning to FIG. 1, one embodiment of an apparatus 10 of the present invention includes a support bar 24 removably secured to the footrest 16 of an item of furniture, such as a podiatry chair, surgical table, pedicure stool, pedicure chair, or manicure table. The support bar 24 can be secured using front-and-back straps 14a and 14b, or side-to-side straps 12a and 12b. The support bar 24 can also be removably secured to the chair using other mechanisms, including but not limited to, hooks, straps, ties, shock cords, hook and loop fasteners, screws, and bolts.

The support bar 24 typically has two recesses 18a and 18b to help position the patient's extremities over the receptacle opening. However, they are not necessary for the operation of the present invention. The recesses 18a and 18b can be adapted to position a patient's arms or legs, for example.

A hoop-shaped frame 20 is secured to the support bar 24. The frame 20 can be removably secured, permanently secured, or unitary with the support bar 24. In FIG. 1, the frame 20 has been merely inserted into holes 22a and 22b in the support bar 24. Other securing mechanisms can be used. In addition, a sleeve 36 may be provided to substantially cover the frame 20, and may be made of plastic, paper, or other material to insulate the patient or client from the frame 20 material. The frame 20 is constructed so that it will hold its shape when flexed. It can be made of a plastic or metal material, and metal wire is preferred.

The frame 20 can be adjustable to change the area 38 enclosed by the frame 20 and support bar 24. Any means of deforming the frame 20 may be used. Also, the holes 22a and 22b can go all the way through the support bar 24 to enlarge or shrink the area 38 enclosed. The frame 20 can also be made wider without changing the area enclosed by the frame. The frame 20 can also be substantially rigid and non-adjustable.

Since some manicure tables already have a support bar 24, the invention may be provided as a frame 20 adapted to be removably secured to the support bar 24, and may also be provided with or without a sleeve 36.

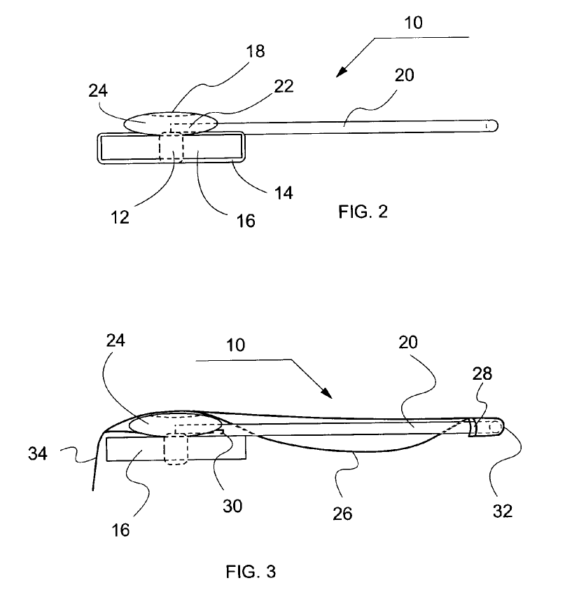

FIG. 2 is a side elevation of the apparatus 10 shown in FIG. 1. Although the frame 20 is shown having a round cross section, it could just as well have any other type of cross section, including square.

FIG. 3 is a side elevation of the apparatus shown it in FIG. 1 and FIG. 2. A receptacle 26 made of flexible material and is removably attached to, or disposed upon, the apparatus 10. The receptacle can be made of plastic or paper material, or other thin and flexible material capable of containing debris, or a combination of the foregoing. The end nearest the practitioner 32 may be held on, in part, by elastic gathers 28. The end nearest the patient or client 34 may be secured using a hook 30, which can be secured or molded to the support bar 24. Equivalent mechanisms and apparatuses for securing the receptacle 26 to the frame 20 include, but are not limited to, folding over and heat sealing, sealing with glue, stapling, press fits with an auxiliary support structure, and using perforations to assist in the removal of the receptacle. The front-and-back straps 14 of FIG. 2 are not shown in FIG. 3 only to make the drawing more clear. The straps 14 may be used in any of the embodiments.

Because the apparatus covers the practitioner's lap, it is also suitable as a receptacle for use in other surgical procedures besides podiatry. It can be used for procedures on any extremity, including hands and arms. It is not only suitable for pedicures, but manicures as well.

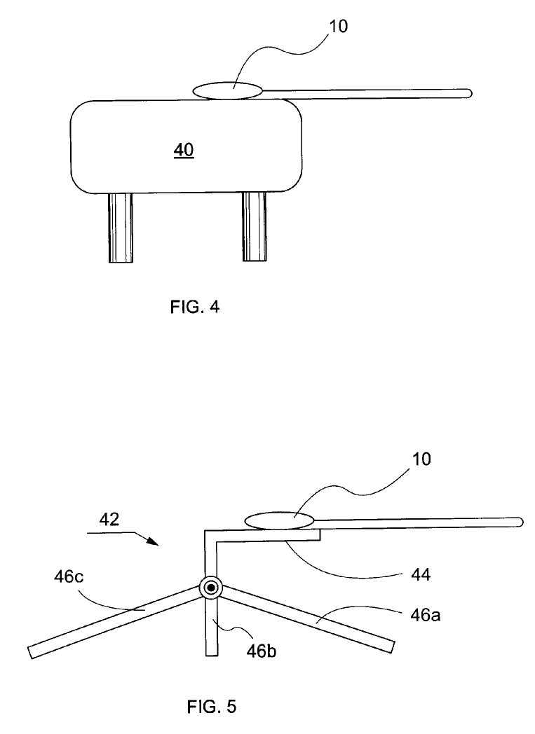

FIG. 4 is a side elevation view of the apparatus 10 used with a stool 40, which is often done with pedicures. One can see that a client can comfortably put her leg over the support bar when seated and using a stool for the support.

FIG. 5 is a side elevation view of the apparatus 10 used with a stand 42, preferably with a plurality of height-adjustable legs shown in this example as 46a, 46b, and 46c. The stand 42 may be one that is commercially available. The apparatus 10 is secured to the portion 44 that is supposed to remain stationary. Examples of commercially available stands include, but are not limited to, medical device stands, music stands, and camera tripods.

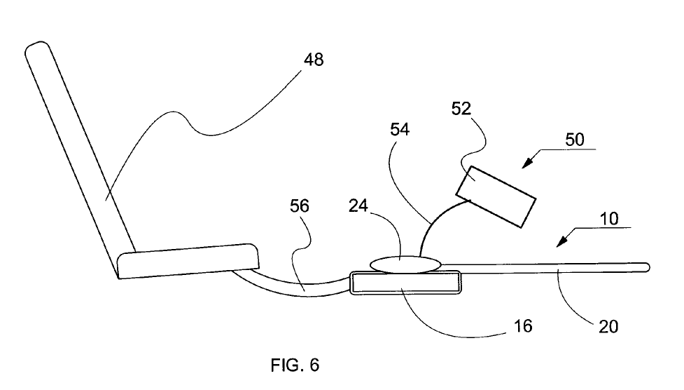

FIG. 6 is a side elevation of the apparatus 10 secured to a podiatric chair 48 with a securing member 56. The apparatus 10, in any embodiment, may also be provided with an examination shield assembly 50, comprising a transparent examination shield 52 secured to a flexible arm 54 that can be secured to the apparatus support bar. Another embodiment would have the flexible frame 20 secured directly to an item of furniture, such as a podiatric chair 48, without a support bar 16.

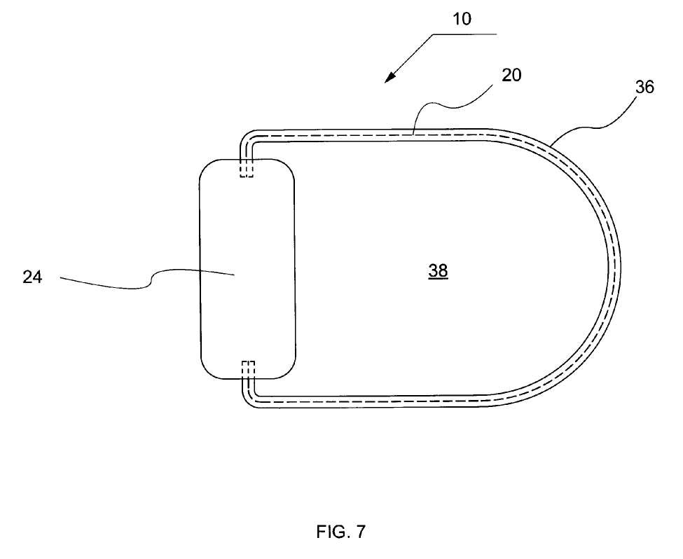

FIG. 7 and subsequent figures show an embodiment of the present invention with a swivel frame 20, where the ends of the frame are bent at a right angle and positioned inside the support bar 24.

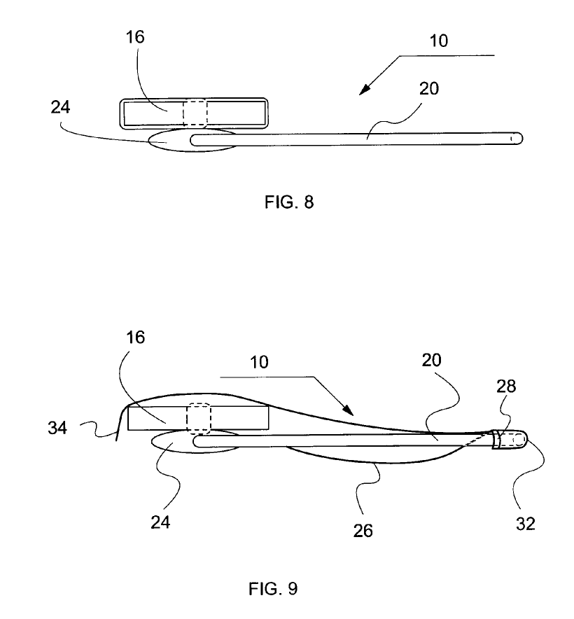

FIG. 8 is a side elevation of the embodiment shown in FIG. 7, which also shows that the apparatus 10 with a swivel frame 20 can be secured under a chair's footrest 16.

FIG. 9 shows the embodiment of FIG. 8 with a receptacle 26 disposed thereon. The end of the receptacle nearest the patient 34 is shown draped over the end of the piece of furniture used.

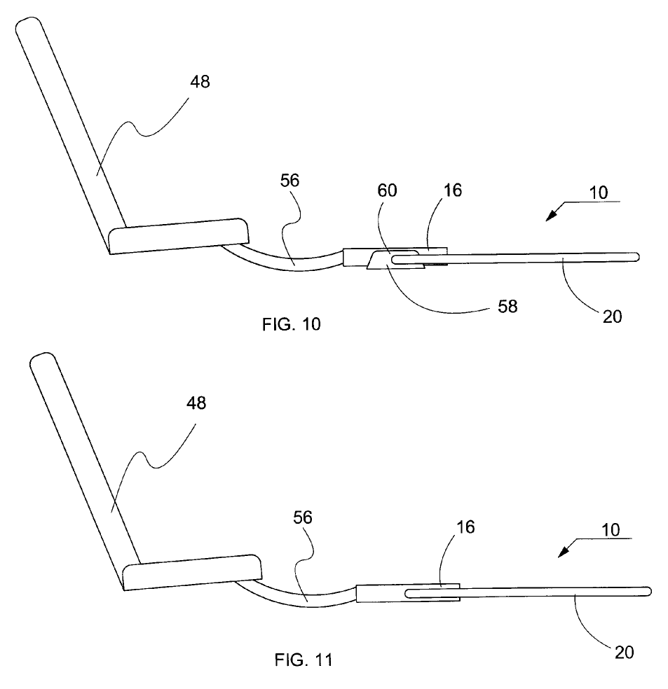

FIG. 10 is a side elevation of the apparatus 10 secured with a securing member 56 to an article of furniture 48. In this embodiment, a different support bar 58 has elongated ends 60 at each end of the support bar 58 that are set substantially perpendicularly to the support bar 58 to wrap around the footrest 16 and provide a higher pivot point for the swivel frame 20.

FIG. 11 is an embodiment of the present invention with the frame 20 directly rotatably secured to the footrest 16 of a podiatric chair 48 without a support bar. The frame 20 does not have to be secured to a support bar to be rotatably secured to an article of furniture.

While there have been described what are at present considered to be the preferred embodiments of this invention, it will be obvious to those skilled in the art that various changes and modifications may be made therein without departing from the invention, and it is, therefore, aimed to cover all such changes and modifications as fall within the true spirit and scope of the invention.

What is claimed is:

1. An apparatus for collecting debris generated from a procedure on a human's extremities comprising:
a support bar adapted to support at least one of a patient's extremities; and
a frame secured to a support bar, the frame adapted to receive at least one receptacle; and
a flexible receptacle.

2. The apparatus of claim 1, wherein the support bar has at least one recess adapted to conform to at least one of a patient's extremities.

3. The apparatus of claim 1 further comprising a fastening mechanism for fastening the support bar to an item of furniture used in the procedure, wherein the fastening mechanism is at least one taken from the group consisting of a hook, strap, tie, shock cord, hook and loop fastener, screw, and bolt.

4. The apparatus of claim 1, wherein the frame is removably secured to the support bar at two locations, and is made of a flexible material adapted to hold its shape when flexed.

5. The apparatus of claim 4, wherein the flexible material is plastic or metal.

6. The apparatus of claim 4, further comprising a sleeve covering the frame.

7. The apparatus of claim 1, wherein the receptacle is made of at least one taken from the group consisting of plastic and paper.

8. The apparatus of claim 7, the receptacle comprising at least one of the group consisting of elastic gathers, perforations, friction fit, and a glued connection.

9. The apparatus of claim 1, further comprising an item of furniture, the item of furniture being one selected from the group consisting of a podiatry chair, surgical table, pedicure stool, pedicure chair, and manicure table.

10. The apparatus of claim 1, further comprising:
a transparent examination shield secured to
an adjustable arm secured to the support frame.

11. The apparatus of claim 1 wherein the frame is substantially rigid.

12. An apparatus for collecting debris generated from a procedure on a human's extremities comprising:
an article of furniture;
a hoop-shaped frame having two opposing ends, the frame rotatably secured to the article of furniture by the two opposing ends; and
a flexible receptacle disposed upon the frame.

13. The apparatus of claim 12, wherein the article of furniture is one selected from the group consisting of a podiatry chair, surgical table, pedicure stool, pedicure chair, and manicure table.

14. The apparatus of claim 12, wherein the frame is made of a material that enables the frame to hold its shape when flexed.

15. The apparatus of claim 14, wherein the frame material is metal or plastic.

16. The apparatus of claim 12, further comprising a sleeve covering the frame.

17. An apparatus for collecting debris generated from a procedure on a human's extremities comprising:
a support bar;
a hoop-shaped frame having two opposing ends, the frame rotatably secured to the support bar by the two opposing ends; and
a flexible receptacle disposed upon the frame.

18. The apparatus of claim 17 further comprising a fastening mechanism for fastening the support bar to an item of furniture used in the procedure, wherein the fastening mechanism is at least one taken from the group consisting of a hook, strap, tie, shock cord, hook and loop fastener, screw, and bolt.

19. The apparatus of claim 17, further comprising a chair leg rest, wherein the support bar is secured underneath the chair leg rest.

20. The apparatus of claim 17, the support bar comprising elongated ends substantially perpendicular to the support bar middle, and wherein the frame ends are secured to the support bar elongated ends.
